(19) United States
(12) Patent Application Publication
(10) Pub. No.: US 2019/0322091 A1
(43) Pub. Date: Oct. 24, 2019

(54) Waterborne Polyurethane Complex and Composition

(71) Applicant: Plastics Industry Development Center, Taichung City (TW)

(72) Inventors: Chi-Hau Fang, Taichung City (TW); Ting-Chun Yu, Taichung City (TW); Meng-Heng Lai, Taichung City (TW); Kuang-Ying Hsueh, Taichung City (TW)

(21) Appl. No.: 16/383,791

(22) Filed: Apr. 15, 2019

(30) Foreign Application Priority Data
Apr. 18, 2018 (TW) 107113255

(51) Int. Cl. B32B 27/40; B32B 7/12; C09J 7/38; C09J 175/04; C09J 11/06; C08G 18/48; C08G 18/22; A61F 13/02

(52) U.S. Cl. CPC B32B 27/40; C08L 75/04; C09J 7/38; C09J 175/04; C09J 11/06; C08G 18/48; C08G 18/227; A61F 13/0213; B32B 2250/02; B32B 2250/04; C08L 2203/02; B32B 2535/00; B32B 2307/7166; B32B 2307/7163; A61F 2210/0004; B32B 7/12

(57) ABSTRACT

A waterborne polyurethane complex and composition comprise a waterborne polyurethane layer being attached to a functional adhesive layer. The waterborne polyurethane layer contains a catalyst with content less than 0.001 wt %, and the catalyst is organic zinc or organic bismuth. The waterborne polyurethane complex is biocompatible and non-irritable, and is adapted for cell therapy or biomedical application.

### FIELD OF INVENTION

The present invention generally relates to a waterborne polyurethane complex that is adapted for biomedical application, and more particularly it relates to a waterborne polyurethane complex and composition for wound care treatment.

The present invention has been developed primarily to provide a dressing for wound care treatment, and will be described hereinafter with reference to this application. However, it will be appreciated that the invention is not limited to this particular field of use.

### BACKGROUND OF THE INVENTION

Traditional wound care products like gauze, bandage or band-aid are used to provide a covering for and around a wound, protecting the wound from harms, contaminations or infections. However, wounds of a patient may have different etiology due physical or thermal injury, or medical conditions (for example, patient whom suffers from diabetes). Depending on the type of wound, suitable wound care products should be applied and used to improve a healing process or condition of the wound. The said traditional wound care products are normally opaque where the healing process of the wound cannot be readily observed without removal.

Further, the said traditional wound care products are unable to provide effective treatments for patients with severe wounds, or for patient whom suffers from diabetic ulcers, serious burns or with lowered level of immunity, and so forth. Nor it does facilitate the healing process or condition for patients with severe wounds or with lowered level of immunity.

Furthermore, many wound dressings are designed to be in contact with the wound. The said traditional wound care products are not biocompatible or biodegradable. As such, a wound dressing for covering the wound and acting as a barrier against contaminations or infections and facilitating the healing process with no or low adverse reactions, irritations or sensitization for the patient is much needed.

The present invention seeks to provide a waterborne polyurethane complex, composition and method for wound care treatment that will overcome or substantially ameliorate at least one or more of the deficiencies of a prior art, or to at least provide an alternative solution to the problems.

It is to be understood that, if any prior art information is referred to herein, such reference does not constitute an admission that the information forms part of the common general knowledge in the art.

### SUMMARY OF THE INVENTION

According to a first aspect of the present invention, a waterborne polyurethane composition for wound care treatment having: a hard segment and a soft segment, wherein: the hard segment is formed with diisocyanate reaction and a first chain extender, the first chain extender for the hard segment include ethylenediamine or p-phenylenediamine; the soft segment is a polyol and a second chain extender, the polyol includes polyether polyol or polyester polyol, the second chain extender for the soft segment is 2,2-bis(hydroxymethyl)propionic acid; a mole ratio between isocyanate functional group of the hard segment and hydroxyl functional group of the soft segment is at a range of 1.70:1 to 1.80:1; and the waterborne polyurethane composition further contains a catalyst with a content of less than 0.001 wt %, wherein the catalyst is organic zinc or organic bismuth.

In accordance with a third aspect of the present invention, further provided is a waterborne polyurethane complex for wound care treatment comprising a waterborne polyurethane layer attached to a functional adhesive layer, wherein the waterborne polyurethane layer is prepared according to the first and second aspect of the present invention. In accordance, the present invention has the following advantages:

1. By selectively identifying a biocompatible material, compositions and adjusting parameters in the production procedures of the waterborne polyurethane, the present invention is highly adaptable, biocompatible and biodegradable for cell-based therapy in patients. In particular, the waterborne polyurethane exhibits a high degree of biocompatibility with stem cells, and is also adapted for other biomedical applications

2. The waterborne polyurethane disclosed by the present invention is highly elastic and it can be easily shaped or expanded to provide a complete coverage to the wound or injured site of the patient.

3. The present invention is also highly transparent. As such, the status and progress of the healing can be easily observed throughout the treatment without the necessity to remove the wound dressing for observation.

Many of the attendant features and advantages of the present invention will become better understood with reference to the following detailed description considered in connection with the accompanying figures and drawings.

### BRIEF DESCRIPTION OF THE DRAWINGS

The steps and the technical means adopted by the present invention to achieve one or more of the above objectives can be best understood by referring to the following detailed description of the preferred embodiments and the accompanying drawings.

FIG. 1 is a test result of biocompatibility of the waterborne polyurethane in accordance to the present invention;

FIG. 2 is a cell morphology of the waterborne polyurethane in accordance to the present invention;

FIG. 3 is a series of bar-diagrams of quantity/numbers and viability of alive adipose-derived stem cells in accordance to the present invention;

FIG. 4 is a microscopic observation of the stem cells proliferation and aggregation in accordance to the present invention;

FIG. 5 is a microscopic observation of the stem cells differentiation ability in accordance to the present invention;

FIG. 6 is a schematic diagram of the wound dressing structure with the waterborne polyurethane in accordance to the present invention;

FIG. 7 is a schematic diagram of a first embodiment of the cell carrier body in accordance to the present invention;

FIG. 8 is a schematic diagram of the second embodiment of the cell carrier body in accordance to the present invention;

FIG. 9 is a schematic diagram of the third embodiment of the cell carrier body in accordance to the present invention; and

FIG. 10 is an illustration of applying the wound dressing complex in accordance to the present invention.

### DETAILED DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts. It is not intended to limit the method by the exemplary embodiments described herein. In the following detailed description, for the purposes of explanation, numerous specific details are set forth in order to provide a thorough understanding of the disclosed embodiments. It will be apparent, however, that one or more embodiments may be practiced without these specific details. As used in the description herein and throughout the claims that follow, the meaning of “a”, “an”, and “the” may include reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the terms “layer” may infer to be a film or a membrane.

[Composition]

The waterborne polyurethane disclosed in present invention includes a main chain of polyurethane comprising a hard segment and a soft segment. The hard segment is formed by reaction of diisocyanate and a first chain extender. The soft segment is formed by a polyol and a second chain extender. By varying the composition of each segment, it provides more subtle control of the waterborne polyurethane structure, complex and properties of the present invention.

With reference to the above, the said diisocyanate could be, but not limited to, aliphatic diisocyanates or alicyclic diisocyanate. The first chain extender for the hard segment includes ethylenediamine (EDA) or p-phenylenediamine (PDA). The polyol is, but not limited to, polyether polyol or polyester polyol. The second chain extender for the soft segment is 2,2-bis(hydroxymethyl)propionic acid (DMPA).

A mole ratio between isocyanate functional group of the hard segment and hydroxyl functional group of the soft segment is at a range of 1.70:1 to 1.80:1, and more preferably, 1.77:1. A higher level of biocompatibility, biodegradability and biological activity is obtained by higher mole ratio of isocyanate functional group. More particularly, the present invention is highly compatible with stem cells. Therefore, it is extremely useful for wound care treatment and other biomedical applications. For example, it could be used as wound dressings or scaffolds in tissue engineering.

In order to facilitate the synthesis of the waterborne polyurethane, a catalyst and a neutralizer may be included in the synthesis procedure. The said catalyst is preferred to be a biocompatible catalyst in a low content, preferably under 0.0015 wt %, and even more preferably under 0.0005 wt %. The catalyst includes organic zinc compound, organic bismuth compound or combination thereof, like T12, C83 and Z22. The said neutralizer is, but not limited to, triethyleneamine (TEA) which is also biocompatible.

Through systematical control and fine turning the compositions and reaction conditions, key components that forms the formulation and characteristics of the waterborne polyurethane and its physicochemical properties is identified, wherein the waterborne polyurethane is adapted for wound care treatment or wound dressing. Thus, the present invention provides an advantage for a wound dressing material that is non-allergic and highly compatible for usage in cell therapy. For example, the waterborne polyurethane with stem cell complex as wound dressing for the patient provides adequate moist environment, facilitation of healing process and prevention of scab formation and dehydration of the wound bed. With reference to chart 1, a range of contents of the aforementioned compositions are provided.

CHART 1

| Material | Name | Content (mole ratio) | Content (Wt %) |
|---|---|---|---|
| Diisocyanate | IPDI/H12MDI | 0.264-4.764 | 15-50 |
| Polyol | PTMEG650/ | 0.070-1.315 | 50-80 |
| | 1000/2000 | | |
| Second chain | DMPA | 0.076-1.374 | 1-5 |
| extender | | | |
| Catalyst | T12/(C83\grave{ }Z22) | 1.0*10⁻⁶-2.0*10⁻⁵ | 0.0001-0.0015 |
| Neutralizer | TEA | 0.470-1.415 | 1-5 |
| First chain | EDA/PDA | 0.088-1.660 | 1-5 |
| extender | | | |

[Production Method]

A production method of the present invention comprises mainly three major phases: a synthesis phase, a prepolymer forming phase and an emulsifying dispersion phase.

The synthesis phase: In the synthesis phase, the polyol is heated to 80° C. for at least 8 hours. The second chain extender for soft segment is added into heated polyol and water from the polyol is removed for 40 to 60 minutes under a vacuum state (around 750˜760 mmHg). After cooling down of the mixture, the heated polyol and the second chain extender to 70° C., the diisocyanate is then added and well blended with the second chain extender for soft segment and the polyol. The catalyst is then added and allowed for reaction under 90˜95° C. After the catalyst is added, a titration method may be performed by adding dibutylamine to test the completion of the synthesis phase.

The processed temperature at 90 to 95° C. for the catalyst is a much higher temperature than the conventional synthesis procedure for catalytic reaction. An advantage of processing the catalyst under the high temperature is that a faster and more completed reaction may be provided in synthesis phase. The content of the catalyst can therefore be reduced to extremely low and subsequently reducing the content of heavy metals from the catalyst to achieve great biocompatibility, providing no or low adverse reactions, irritations or sensitization for usage in cell therapy, and in particular is adapted for use in stem cell therapy for wound care treatment.

The prepolymer forming phase: After the titration method is finished, the mixture is cooled to approximately 50 to 80° C. A methyl ethyl ketone/acetone may be added to adjust viscosity of the mixture during the cooling process. After the viscosity reached a desire condition, the neutralizer is added and allowed for reaction for approximately 30 minutes and a waterborne polyurethane prepolymer is obtained.

The emulsifying dispersion phase: The waterborne polyurethane prepolymer is dispersed by blending at 1000 to 2000 RPM. During the blending, deionized water (DI) water is added to disperse the waterborne polyurethane prepolymer under hydration condition, and a solvent of the first chain extender for the hard segment is added to extend the main chain of the waterborne polyurethane. After a further blending of the mixture for an hour, the waterborne polyurethane, the present invention, is then successfully obtained.

### EXAMPLES

### Example 1

| Material | Name | Content (mole ratio) | Content (Wt %) |
|---|---|---|---|
| Diisocyanate | IPDI | 0.271 | 26 |
| Polyol | PTMEG2000 | 0.075 | 64 |
| Second chain | DMPA | 0.078 | 4 |
| extender | | | |
| Catalyst | T12 | 1.9*10⁻⁶ | 0.0005 |
| Neutralizer | TEA | 0.08 | 3 |
| First chain | EDA | 0.1 | 3 |
| extender | | | |

### Example 2

| Material | Name | Content (mole ratio) | Content (Wt %) |
|---|---|---|---|
| Diisocyanate | IPDI | 0.385 | 17 |
| Polyol | PTMEG650 | 0.191 | 78 |
| Second chain | DMPA | 0.076 | 2 |
| extender | | | |
| Catalyst | C83 | 9.7*10⁻⁶ | 0.0014 |
| | Z22 | 1.6*10⁻⁵ | 0.0014 |
| Neutralizer | TEA | 0.078 | 2 |
| First chain | PDA | 0.1 | 1 |
| extender | | | |

### Example 3

| Material | Name | Content (mole ratio) | Content (Wt %) |
|---|---|---|---|
| Diisocyanate | H12MDI | 0.368 | 41 |
| Polyol | PTMEG650 | 0.175 | 49 |
| Second chain | DMPA | 0.076 | 4 |
| extender | | | |
| Catalyst | C83 | 4.4*10⁻⁶ | 0.0014 |
| | Z22 | 7.4*10⁻⁶ | 0.0014 |
| Neutralizer | TEA | 0.078 | 3 |
| First chain | PDA | 0.1 | 3 |
| extender | | | |

### Example 4

| Material | Name | Content (mole ratio) | Content (Wt %) |
|---|---|---|---|
| Diisocyanate | H12MDI | 0.264 | 30 |
| Polyol | PTMEG2000 | 0.07 | 60 |
| Second chain | DMPA | 0.076 | 4 |
| extender | | | |
| Catalyst | C83 | 1.5*10⁻⁶ | 0.0005 |
| | Z22 | 2.5*10⁻⁶ | 0.0005 |
| Neutralizer | TEA | 0.078 | 3 |
| First chain | EDA | 0.1 | 3 |
| extender | | | |

### Example 5

| Material | Name | Content (mole ratio) | Content (Wt %) |
|---|---|---|---|
| Diisocyanate | IPDI | 0.385 | 37 |
| Polyol | PTMEG650 | 0.191 | 53 |
| Second chain | DMPA | 0.076 | 4 |
| extender | | | |
| Catalyst | C83 | 4.4*10⁻⁶ | 0.0014 |
| | Z22 | 7.4*10⁻⁶ | 0.0014 |
| Neutralizer | TEA | 0.078 | 3 |
| First chain | EDA | 0.1 | 3 |
| extender | | | |

### Example 6

| Material | Name | Content (mole ratio) | Content (Wt %) |
|---|---|---|---|
| Diisocyanate | IPDI | 0.33 | 31 |
| Polyol | PTMEG1000 | 0.137 | 58 |
| Second chain | DMPA | 0.076 | 4 |
| extender | | | |
| Catalyst | C83 | 3.9*10⁻⁶ | 0.0012 |
| | Z22 | 6.5*10⁻⁶ | 0.0012 |
| Neutralizer | TEA | 0.078 | 3 |
| First chain | EDA | 0.1 | 3 |
| extender | | | |

A series of tests are provided with control groups for comparison in examples 1˜5 for showing the waterborne polyurethane's biocompatibility and non-adverse response to stem cells.

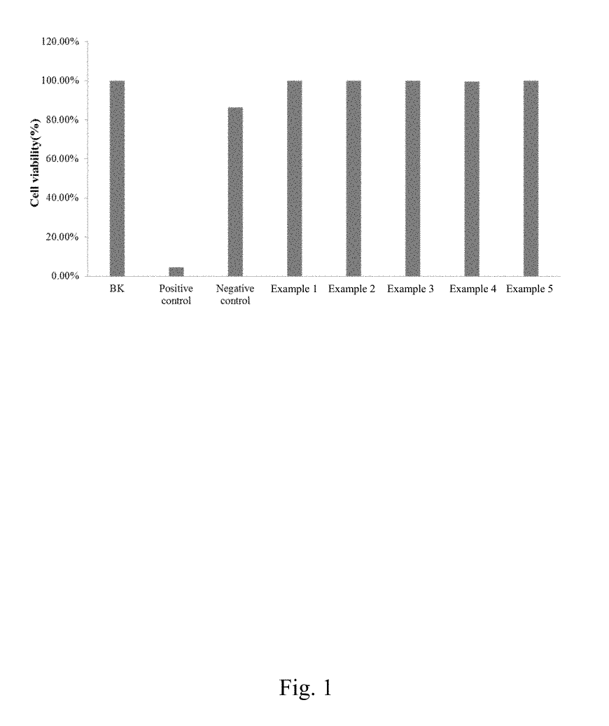

With reference to FIG. 1, a test result of biocompatibility is shown. BK sample in FIG. 1 is a plain control group without any material being applied to cultured cells. Positive control sample in FIG. 1 is another control group containing 100% of zinc diethyldithiocarbamate (ZDEC) for cells culture. Negative control sample in FIG. 1 is also a control group containing waterborne polyurethane but reacted with tin catalyst. As shown in FIG. 1, cell viability of the control groups (BK sample, positive control sample, negative control sample) are 100%, 4.5% and 86.3%, respectively. Positive control sample shows obvious cytotoxicity. Negative control sample also exhibited lowered cell viability which suggests that tin catalyst is harmful to the cells. The cell viability of the examples 1˜5 of the present invention are 100%, 100%, 100%, 99.7% and 100%. Compared to all control groups, the present invention has great biocompatibility.

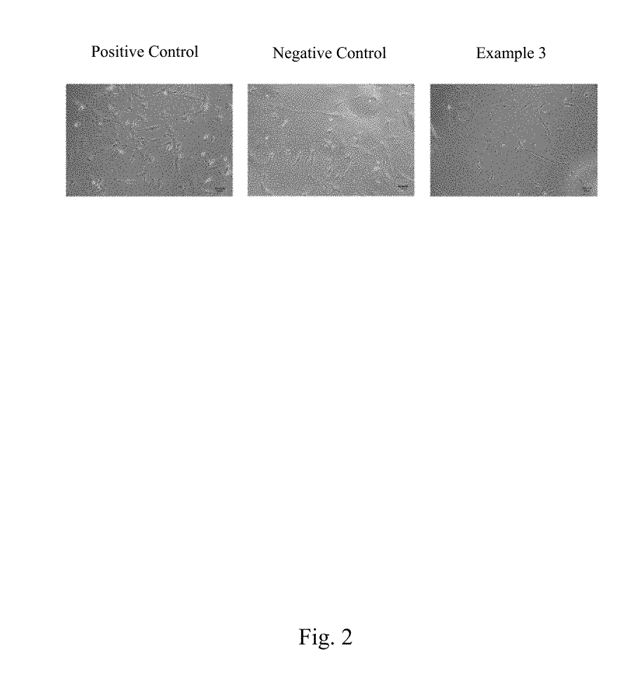

With reference to FIG. 2, cell morphology under a microscope is provided. Adipose-derived stem cells are cultured with the negative control sample, the positive control sample and example 3 of the present invention for 24 hours. The results are observed by a 100× microscope. As shown in FIG. 2, adipose-derived stem cells appear in all tested groups. Cells density of the positive control sample is higher than cells density of the negative control sample and example 3 of the present invention. The cell morphology results showed non-obvious difference between the negative control sample and example 3 of the present invention.

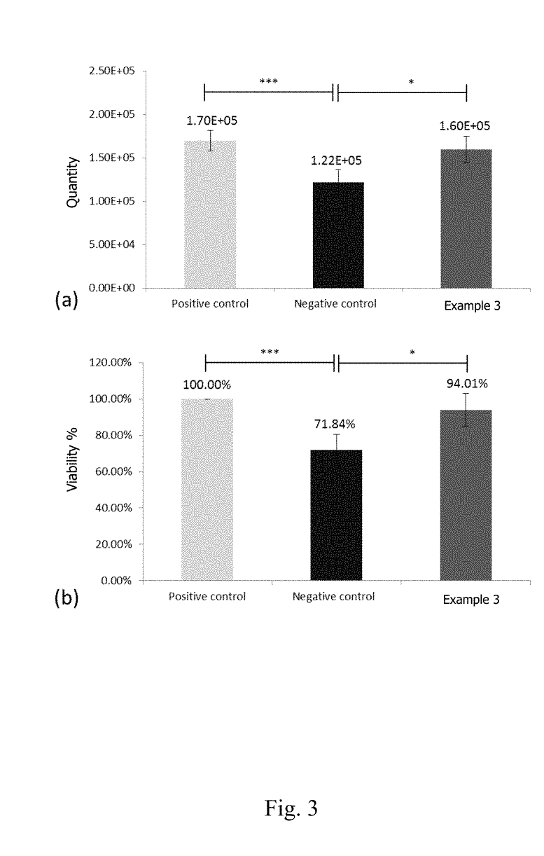

FIGS. 3 to 5 are a series of stem cells testing results showing stem cells biocompatibility and decreased level or lowered level of sensitization response to the present invention. With reference to FIG. 3, adipose-derived stem cells are cultured with the negative control sample, the positive control sample and example 3 of the present invention, respectively. FIG. 3(a) shows quantity/numbers of alive adipose-derived stem cells and FIG. 3(b) shows viability of adipose-derived stem cells after 24 hours of cultivation corresponded to FIG. 3(a). “*” symbol in FIG. 3 represents p value <0.05 and “***” symbol in FIG. 3 represents p value <0.01 which can be considered a significant difference in statistics. FIG. 3 shows quantity/numbers of alive adipose-derived stem cells of positive control sample are 1.7×10⁵ cells. Quantity/numbers of alive adipose-derived stem cells of the negative control sample are 1.22×10⁵ cells and quantity/numbers of alive adipose-derived stem cells of example 3 are 1.6×10⁵ cells. The result of the negative control sample shows obvious decrease in numbers of alive adipose-derived stem cells (p value only=0.0017). The result of example 3 of the present invention otherwise shows improved condition of numbers of alive adipose-derived stem cells (p value=0.388).

The viability of adipose-derived stem cells disclosed in FIG. 3(b), where the viability of the negative control is 71.84% (p value=0.0035) and the viability of example 3 is 94.01% (p value=0.185) which also shows that the present invention has greater stem cells viability.

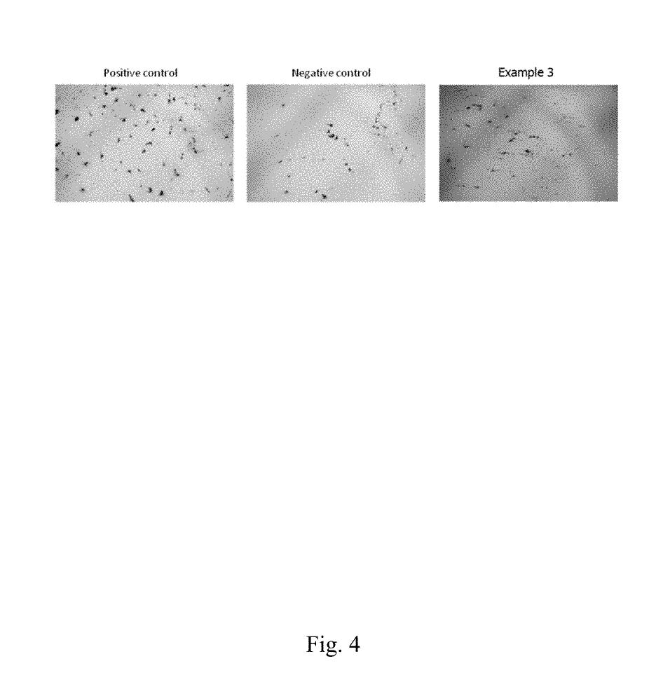

With reference to FIG. 4, adipose-derived stem cells are cultured with the negative control sample, the positive control sample and example 3 of the present invention under a low cultivate density for 14 days, respectively. After staining by giemsa stain, cell nucleus of adipose-derived stem cells will become light blue from violet. The result of the stem cells proliferation and aggregation can then be observed by a 40× electron microscope. As shown in FIG. 4, stem cells proliferation ability of example 3 is better than stem cells proliferation ability of the negative control sample.

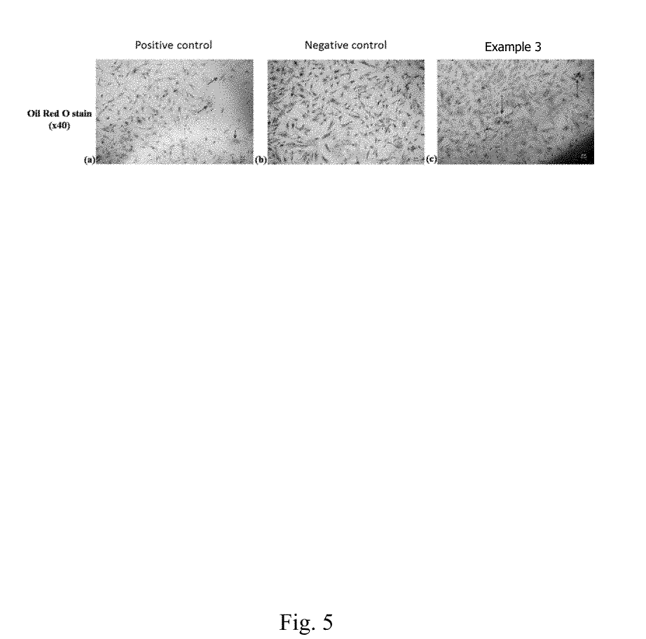

With reference to FIG. 5, adipose-derived stem cells are cultured, and osteogenic is induced to the negative control sample, the positive control sample and example 3 of the present invention for 14 days, respectively. Cell differentiation ability is analyzed by Oil Red O staining and is observed by a 40× electron microscope. As shown in FIG. 5, the Oil Red O stain successfully stained to cytoplasm of the stem cells. The cell differentiation ability of the present invention is better than the cell differentiation ability of the negative control sample.

A thin film made by the present invention with thickness of only 20˜50 μm is tested showing great mechanical properties of the present invention, including fracture strength, elongation rate, moisture permeability and transparency as shown in chart 2.

CHART 2

| Properties | Value |
|---|---|
| Fracture Strength (kgf/cm²) | 90~250 |
| Elongation rate (%) | 230~340 |
| Moisture permeability (g/m² · d) | 500~1200 |
| Transparency (%) | >90 |

[Application/Complex]

With reference to FIG. 6, a waterborne polyurethane wound dressing complex 10 of the present invention is provided. The wound dressing complex 10 has at least two layers being connected together. A first layer is made with the waterborne polyurethane as disclosed (hereinafter “the waterborne polyurethane layer” 11) and a second layer is a cell carrier body 13. The aforementioned layers may infer to be a film or a membrane.

A pressure sensitive adhesive layer 12 and a functional adhesive layer 14 may be further applied between the waterborne polyurethane layer 11 and the cell carrier body 13 for a differential or securer adhesion. The pressure sensitive adhesive layer 12 is adhered to a top surface 111 of the waterborne polyurethane layer 11 (where a bottom surface of the waterborne polyurethane layer is referenced as 113 in FIG. 6). The functional adhesive layer 14 is adhered to a bottom surface 133 of the cell carrier body 13 (where a top surface of the cell carrier body is referenced 131 in FIG. 6). The pressure sensitive adhesive layer 12 and the functional adhesive layer 14 are therefore adhered together. More preferably, a size or surface of the cell carrier body 13 is same as the size or surface of the functional adhesive layer 14 but smaller than the size or surface of the pressure sensitive adhesive layer 12 and the waterborne polyurethane layer 11. Therefore, the wound dressing complex 10 is able to be adhered to peripheral parts on the skin or body of the patient's wound or injured site by the pressure sensitive adhesive layer 12. The pressure sensitive adhesive layer 12 may be an acrylic adhesive.

The waterborne polyurethane layer 11 is preferably a film or a membrane with high elasticity, high biocompatibility to body tissues, cells or stems cells, high moisture vapor permeability and transparency. Further, by adjusting the composition or formula, reaction time and temperature in synthesizing the waterborne polyurethane layer 11, the biodegradability of the waterborne polyurethane layer 11 may also be improved such that is adapted for wound care treatment or other biomedical application purposes including, but not limited to, ophthalmology, dentistry, implant or drug delivery.

The functional adhesive layer 14 is preferably to have different adhesiveness between the pressure sensitive adhesive layer 12 and the cell carrier layer 13. The adhesion strength between the functional adhesive layer 14 and the pressure sensitive adhesive layer 12 are stronger than the adhesion between the functional adhesive layer 14 and the cell carrier body 13 to allow the cell carrier body 13 be readily removed or detachable from the functional adhesive layer 14. Preferably, the functional adhesive layer 14 is also highly biocompatible. It may be an oily gel, an aqueous gel or silica gel contained functional materials. The functional materials may further provide absorption of tissue fluid, antiseptic, release of growth factor or to provide suitable nutrition to the cell carrier body 13 or stem cells to help facilitate wound recovery. Depending on the type of the functional material, the corresponding material of the functional adhesive layer 14 is then selected. For example, when choosing an oily based functional material, the functional adhesive layer 14 is better adapted for the oily gel like polypropylene gel (PE gel or PE adhesive) to increase its compatibility. By the same token, the aqueous gel is more adapted or suitable for water based functional material.

With reference to FIG. 7, the cell carrier body 13 can carry or transport stem cells 15 thereon. The stem cells 15 may be adipose-derived stem cells or mesenchymal stem cells with abilities to facilitate or accelerate wound healing. The cell carrier body 13 is preferred to have biocompatibility and biodegradability with the stem cells 15. As shown in FIG. 7, a first embodiment of the cell carrier body 13 comprising one or more membranes, wherein the one or more membranes may be adhered to each other. 135. The stem cells 15 are attached to a surface of the membrane 135.

The functional adhesive layer 14 of the present invention is highly biocompatible, thus the stem cells 15 can also be selectively applied on the functional adhesive layer 14 of the wound dressing complex or structure 10.

With reference to FIG. 8, a second embodiment of the cell carrier body 13 is presented in a film-like foam structure 137 with a porous body 139. An average size of the pores is at a range of 50˜300 um with a porosity above 80%. The porous body 139 can act as a cell-scaffold complex for stem cells 15 attachment to provide an improved condition for its growth, proliferation and differentiation.

With reference to FIG. 9, a third embodiment of the cell carrier body 13 is presented with aqueous gel G. By blending the stem cells 15 with the aqueous gel G, the mixture can be coated on a surface of the waterborne polyurethane layer 11 and directly applied to the wound site of the patient. A preferred embodiment is to blend the aqueous gel G with the stem cells 15 and placing a suspended solution of stem cells 15 into a centrifuge tube containing a measured amount of aqueous gel G. The aqueous gel G may be a natural material or an artificial material with concentration of solid contain in a range of 0.5 to 3%. The aqueous gel G can also be a cell-scaffold complex for stem cells 15 attachment to prolong and retain the stem cells at the wound or injured site. Another way of applying the aqueous gel G stem cells 15 mixture is to apply the mixture directly to the wound or injured site of the patient and then further covering it with the waterborne polyurethane layer 11 of the present invention to create a closure environment preventing outside contamination.

The natural material of the aqueous gel G contains chitin, chitosan, fibrin, collagen, gelatin, hyaluronic acid (HA), alginate or cellulose. The artificial material of the aqueous gel G contains polylactate (PLA), polyorthoester (POE), polycaprolactone (PCL), polyanhydride (PHA), polyoxymethylene (POM) or polyglycolate (PGA) and its copolymer like poly-D,L-lactide-co-glycolide (PLGA).

With reference to FIG. 10, an illustration of applying the wound dressing complex 10 of present invention is provided. The cell carrier body 13 is directly applied to a wound site W of the patient's arm H. The cell carrier body 13 is adapted to fit the wound site W, and the waterborne polyurethane layer 11 and the pressure sensitive adhesive layer 12 is adhered to skin around the wound site W, covering and sealing the wound site W. When the wound dressing complex 10 is required to be replaced or removed, the functional adhesive layer 14, the pressure sensitive adhesive layer 12 and the waterborne polyurethane layer 11 can be easily peeled off separately from the cell carrier body 13 and away from the wound or injured site of the patient due to the designed adhesion differences between the layers. The cell carrier body 13 remain still with the wound W to prevent new born cells or tissues being torn apart or to cause further damages from replacement.

The above specification, examples, and data provide a complete description of the present disclosure and use of exemplary embodiments. Although various embodiments of the present disclosure have been described above with a certain degree of particularity, or with reference to one or more individual embodiments, those with ordinary skill in the art could make numerous alterations or modifications to the disclosed embodiments without departing from the spirit or scope of this disclosure.

What is claimed is:

1. A waterborne polyurethane composition comprising: a main chain of polyurethane including a hard segment and a soft segment, wherein
the hard segment is formed by reaction of diisocyanate and a first chain extender, the first chain extender for the hard segment include ethylenediamine or p-phenylenediamine;
the soft segment is a polyol and a second chain extender, the polyol includes polyether polyol or polyester polyol, the second chain extender for the soft segment is 2,2-bis(hydroxymethyl)propionic acid;
a mole ratio between isocyanate functional group of the hard segment and hydroxyl functional group of the soft segment is at a range of 1.70:1 to 1.80:1; and
the waterborne polyurethane further contains a catalyst with content less than 0.001 wt %, and wherein the catalyst is organic zinc or organic bismuth.

2. The waterborne polyurethane composition as claimed in claim 1, wherein the content of the catalyst is less than 0.0005 wt %.

3. The waterborne polyurethane composition as claimed in claim 1, wherein the waterborne polyurethane is a film or a membrane.

4. The waterborne polyurethane composition as claimed in claim 2, wherein the waterborne polyurethane is a film or a membrane.

5. A waterborne polyurethane complex, comprising a waterborne polyurethane layer attached to a functional adhesive layer, wherein:
the waterborne polyurethane layer comprises a main chain of polyurethane including a hard segment and a soft segment;
the hard segment is formed by reaction of diisocyanate and a first chain extender, the first chain extender for the hard segment include ethylenediamine or p-phenylenediamine;
the soft segment is a polyol and a second chain extender, the polyol includes polyether polyol or polyester polyol, the second chain extender for the soft segment is 2,2-bis(hydroxymethyl)propionic acid;
a mole ratio between isocyanate functional group of the hard segment and hydroxyl functional group of the soft segment is at a range of 1.70:1 to 1.80:1; and
the waterborne polyurethane further contains a catalyst with a content of less than 0.001 wt %, and wherein the catalyst is organic zinc or organic bismuth.

6. The complex or structure as claimed in claim 5, wherein stem cells are attached to the functional adhesive layer.

7. The complex as claimed in claim 5, wherein a cell carrier body with stem cells is further attached to a portion of the functional adhesive layer; and the cell carrier body is biocompatible.

8. The complex as claimed in claim 7, wherein:
a pressure sensitive adhesive layer is included between the waterborne polyurethane layer and the functional adhesive layer; and
adhesion strength between the functional adhesive layer and the pressure sensitive adhesive layer is stronger than the adhesion strength between the functional adhesive layer and the cell carrier body to allow the cell carrier body be readily removed or detachable from the functional adhesive layer.

9. The complex as claimed in claim 7, wherein: the cell carrier body is a membrane and is attached to a portion of the functional adhesive layer.

10. The complex as claimed in claim 8, wherein: the cell carrier body is a membrane and is attached to a portion of the functional adhesive layer.

11. The complex as claimed in claim 7, wherein the cell carrier body is a film-like foam with a porous body; and an average size of the pores is at a range of 50˜300 um with a porosity above 80%.

12. The complex as claimed in claim 8, wherein the cell carrier body is a film-like foam with a porous body; and an average size of the pores is at a range of 50˜300 um with a porosity above 80%.

13. The complex as claimed in claim 7, wherein the cell carrier body is an aqueous gel; and the aqueous gel is a natural material or an artificial material.

14. The complex as claimed in claim 8, wherein the cell carrier layer is an aqueous gel; and the aqueous gel is a natural material or an artificial material.

15. The complex as claimed in claim 5, wherein the functional adhesive layer is biocompatible and is an oily gel, an aqueous gel or a silica gel containing functional materials; and the functional materials have fluid absorption ability and contain anti-bacteria agent or growth factors.
